(19) United States
(12) Patent Application Publication
(10) Pub. No.: US 2026/0262961 A1
(43) Pub. Date: Sep. 10, 2026

(54) BIOIMPEDANCE MEASUREMENT CIRCUIT, METHOD AND ELECTRONIC DEVICE

(71) Applicant: AMS-OSRAM AG, Premstaetten (AT)

(72) Inventors: Predrag MICAKOVIC, Erfurt (DE); Parvathy SASIKALA JAYACHANDRAN PILLAI, Erfurt (DE); Julio SALDANA, Jena (DE)

(21) Appl. No.: 19/167,391

(22) PCT Filed: Feb. 28, 2024
(86) PCT No.: PCT/EP2024/055118

§ 371 Date: Sep. 22, 2025

(30) Foreign Application Priority Data
Mar. 24, 2023 (DE) 10 2023 107 497.0

(51) Int. Cl. A61B 5/0531; A61B 5/00; A61B 5/1495

(52) U.S. Cl. CPC A61B 5/0531; A61B 5/1495; A61B 5/7225; A61B 2560/0223

(57) ABSTRACT

A bioimpedance measurement circuit for determining a corrected body impedance includes a set of terminals, a compensation impedance, a control circuit and an evaluation circuit. The control circuit is configured, in a calibration mode, to control measurement of a first bodypart impedance. Stimulus current is applied through the first and the third terminal and the compensation impedance, and the input voltage is measured between the first and the third terminal, to control measurement of a second bodypart impedance. The stimulus current is applied through the second and the fourth terminal, and the input voltage is measured between the second and the fourth terminal. The control circuit is configured to adjust the compensation impedance based on a difference between the first bodypart impedance and the second bodypart impedance until a calibration criterion is met. The control circuit is configured, in a regular mode, to control measurement of a body impedance.

### CROSS-REFERENCE TO RELATED APPLICATIONS

This application is a US National Stage, filed under 35 U.S.C. § 371, of International Application PCT/EP2024/055118, filed Feb. 28, 2024 and further claims priority to German Application 10 2023 107 479.0, filed on Mar. 24, 2023, the entire content of the above-listed applications are incorporated herein by reference.

### TECHNICAL FIELD

The disclosure relates to a bioimpedance measurement circuit for measuring a bioimpedance across measurement electrodes, and to an electronic device with such bioimpedance measurement circuit. The disclosure further relates to a corresponding bioimpedance measurement method and to a computer program product thereof.

### BACKGROUND

Bioimpedance measurement is a biomedical technique to determine the electrical behavior of living tissue by using an electrode arrangement for applying a stimulus current to a sample, for example a part of a human body, and measure the resulting voltage through a four point measurement method.

The amplitude and phase of the resulting voltage signal will depend on the BIOZ impedance. The bioimpedance of interest may be determined by the measured voltage divided by the stimulus current injected into the tissue.

Due to asymmetries in a current path of the arrangement of the measuring electrodes to perform the four point measurement method, frequency dependent errors are contained in the measurement results. Those asymmetries arise because of a mismatch between the skin/electrode contact impedances. As a result, the measured magnitude of the total input differential signal which is applied to the differential amplifier is different to the expected one and the phase is also affected.

### SUMMARY

Embodiments of the present disclosure provides an improved bioimpedance measurement concept which attenuates effects of mismatch between the skin/electrode contact impedances.

Bioimpedance measurement usually is performed by applying a current to the sample and measure the generated voltage through a 4 point measurement method. The applied current is an alternating current, for example a 50 KHz sine wave.

The amplitude and phase of the resulting voltage signal will depend on the bioimpedance or, in general, body impedance.

The improved bioimpedance measurement concept is based on the finding that the actual measurement of the body impedance is affected by frequency dependent errors due to unavoidable mismatch in contact impedances resulting e.g. in common mode signals. Such mismatch can result from parasitic impedances or capacitances in the signal paths. The improved bioimpedance measurement concept compensates for such mismatch by providing a compensation impedance formed from at least one of first compensation impedance connected to the first terminal and a second compensation impedance connected to the third terminal. The compensation impedance, i.e., impedance values of the first and/or the second compensation impedance, can be adjusted with the aim of minimizing the mismatch. For example, with a calibration before each measurement phase, an adjustment of the compensation impedance can be performed based on a measured difference between the contact impedances. Hence at least two bodypart impedance measurements are performed, the bodyparts e.g. corresponding to a location, where the electrodes are applied, like a wrist and a finger, in case of e.g. usage in a wristband or watch on a wrist.

In an embodiment of a bioimpedance measurement circuit according to the improved bioimpedance measurement concept the bioimpedance measurement circuit comprises a set of terminals comprising a first terminal for connecting a first electrode, a second terminal for connecting a second electrode, a third terminal for connecting a third electrode and a fourth terminal for connecting a fourth electrode, each electrode to be attached to a body. The bioimpedance measurement circuit further comprises a compensation impedance formed from at least one of a first compensation impedance connected to the first terminal and a second compensation impedance connected to the third terminal. A control circuit is included to control application of a stimulus current with a measurement frequency, in particular an AC current, through a first subset of two selected terminals of the set of terminals and measurement of an input voltage in response to the stimulus current at a second subset of two selected terminals of the set of terminals. An evaluation circuit is included for determining a measured impedance in response to the stimulus current and the measured input voltage.

The bioimpedance measurement circuit is configured to be operated at least in a calibration mode of operation and in a regular mode of operation.

For example, in the calibration mode of operation, the control circuit is configured to control measurement of a first bodypart impedance, where the stimulus current is applied through the first and the third terminal and the compensation impedance, and the input voltage is measured between the first and the third terminal, and of a second bodypart impedance, where the stimulus current is applied through the second and the fourth terminal, and the input voltage is measured between the second and the fourth terminal. Furthermore, in the calibration mode of operation, the control circuit is configured to adjust the compensation impedance based on a difference between the first bodypart impedance and the second bodypart impedance until a calibration criterion is met.

Furthermore, in the regular mode of operation, the control circuit is configured to control measurement of a body impedance, where the stimulus current is applied through the first and the second terminal, and the input voltage is measured between the third and the fourth terminal.

Hence with the additional measurements, i.e. of the bodypart impedances in the calibration mode of operation preceding an actual measurement, a more accurate result of the body impedance can be achieved.

For example, the compensation impedance comprises at least one of a resistive portion and a capacitive portion. In particular, the first and the second compensation impedance, whichever is present, each can have a resistive portion and a capacitive portion. For example, during measurement both in the calibration mode of operation and the regular mode of operation, the stimulus current flows through the respective resistive portion(s) while the capacitive portion connects the current path to ground or any other potential.

In various implementations the compensation impedance is digitally controllable, and the control circuit is configured to adjust the compensation impedance with a digital control word.

The calibration criterion may be a number of iterations or measurements in the calibration mode of operation. In addition or as an alternative, the calibration criterion may be a difference threshold for the difference between the first bodypart impedance and the second bodypart impedance.

Further calibration criteria are not excluded by these examples.

For example, the control circuit is configured, in the calibration mode of operation, to perform one of a successive approximation approach and a binary search approach.

In various implementations the bioimpedance measurement circuit is configured to perform a measurement cycle with a plurality of consecutive measurements in the regular mode of operation, wherein each measurement cycle is preceded by a calibration in the calibration mode of operation.

In various implementations the bioimpedance measurement circuit further comprises a signal processing circuit for determining, from the corresponding measurements, the body impedance, the first bodypart impedance and the second bodypart impedance.

The electrode arrangement including the first electrode, the second electrode, the third electrode and the fourth electrode respectively connected to the corresponding terminal of the set of terminals may be part of the bioimpedance measurement circuit.

In an embodiment of a bioimpedance measurement method according to the improved bioimpedance measurement concept the bioimpedance measurement method is performed with a set of terminals comprising a first terminal connected to a first electrode being attached to a body, a second terminal connected to a second electrode being attached to the body, a third terminal connected to a third electrode being attached to the body, a fourth terminal connected to a fourth electrode being attached to the body and with a compensation impedance formed from at least one of a first compensation impedance connected to the first terminal and a second compensation impedance connected to the third terminal. The method comprises:
generating a stimulus current with a measurement frequency;
determining, in a calibration mode of operation, a first bodypart impedance in response to the stimulus current and the measured input voltage, where the stimulus current is applied through the first and the third terminal and the compensation impedance, and the input voltage is measured between the first and the third terminal;
determining, in the calibration mode of operation, a second bodypart impedance in response to the stimulus current and the measured input voltage, where the stimulus current is applied through the second and the fourth terminal, and the input voltage is measured between the second and the fourth terminal;
adjusting, in the calibration mode of operation, the compensation impedance based on a difference between the first bodypart impedance and the second bodypart impedance until a calibration criterion is met; and
determining, in a regular mode of operation, a body impedance in response to the stimulus current and a measured input voltage in response to the stimulus current, where the stimulus current is applied through the first and the second terminal, and the input voltage is measured between the third and the fourth terminal.

The bioimpedance measurement method may be a computer implemented method that e.g. is carried out by a processor or programmable circuit.

Further implementations of the method become readily apparent for the skilled reader from the various implementations described above in conjunction with the bioimpedance measurement circuit.

According to one embodiment of the improved bioimpedance measurement concept, a computer program product is disclosed, the computer program product comprising instructions which, when executed on one or more processors in connection with a first, a second, a third, a fourth terminal and a compensation impedance, e.g. as described above, cause the one or more processors to perform the bioimpedance measurement method according to one of the disclosed implementations.

The various implementations of the bioimpedance measurement circuit and method may be used in various applications and products, e.g. electronic devices, like vital sign monitoring in wearables (smartwatches) or healthcare applications like diagnostics. The body impedance can for example be the basis for determination of Body-Cell-Mass Composition (BCM), hydration level detection, calories consumption, fat, muscle percentage, stress level, to name only a few.

### BRIEF DESCRIPTION OF DRAWINGS

The improved bioimpedance measurement concept will be explained in more detail in the following with the aid of the drawings. Elements and functional blocks having the same or similar function bear the same reference numerals throughout the drawings. Hence their description is not necessarily repeated in the following drawings.

In the drawings:

FIG. 1 shows an example implementation of a bioimpedance measurement circuit;

FIG. 2 shows a further example implementation of a bioimpedance measurement circuit;

FIG. 3 shows various measurement configurations of a bioimpedance measurement circuit;

FIG. 4 shows a further example implementation of a bioimpedance measurement circuit;

FIG. 5 shows an example block diagram of a bioimpedance measurement method; and

FIG. 6 shows a further example implementation of a bioimpedance measurement circuit.

### DETAILED DESCRIPTION

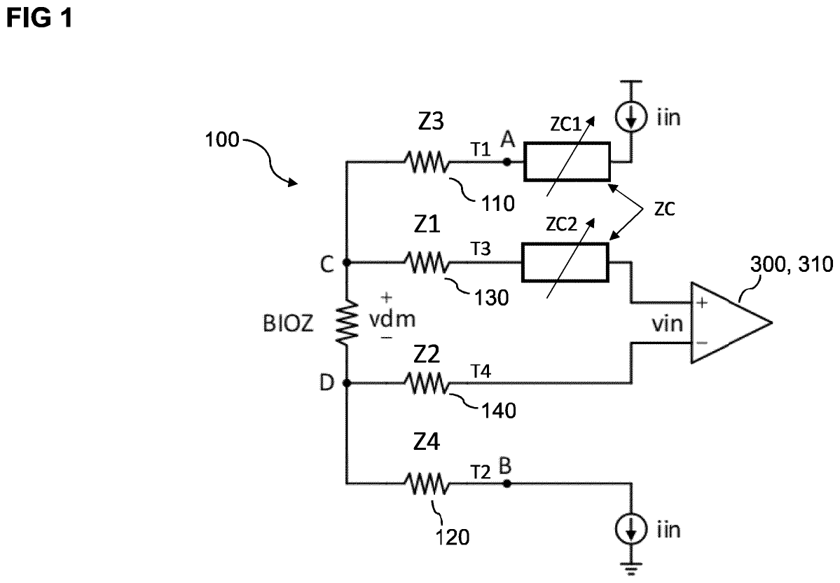

FIG. 1 illustrates an example implementation of a bioimpedance measurement circuit that is based on a four point measurement method. The bioimpedance measurement circuit comprises an electrode arrangement 100 for attaching to a human body. The electrode arrangement 100 includes a first pair of electrodes 110, 120 connected to a first and a second terminal T1, T2 of the bioimpedance measurement circuit for applying a stimulus current iin. The electrodes 110, 120 are attached to nodes A and B of a human body. It is assumed that electrode 110 has an electrode contact impedance Z3, and electrode 120 has an electrode contact impedance Z4.

The electrode arrangement further comprises a second pair of electrodes 130, 140 connected to a third and a fourth terminal T3, T4 of the bioimpedance measurement circuit for measuring a voltage vin between the electrodes 130, 140 of the second pair. The electrodes 130, 140 are attached to nodes C and D of a human body. It is assumed that electrode 130 has an electrode contact impedance Z1, and electrode 140 has an electrode contact impedance Z2.

The bioimpedance measurement circuit further comprises a compensation impedance ZC that is generally formed of a first compensation impedance ZC1 and second compensation impedance ZC2. In some implementations only one of the first and the second compensation impedance ZC1, ZC2 is present. The first compensation impedance ZC1 is connected to the first terminal T1 or electrode 110, in particular is connected between the first terminal T1 or electrode 110 and the upper current source providing the stimulus current iin. The second compensation impedance ZC2 is connected to the third terminal T3 or electrode 130, in particular is connected between the third terminal T3 or electrode 130 and the upper, non-inverting input of amplifier 310. The first and the second compensation impedance ZC1, ZC2 are adjustable.

The electrodes 110, 120, 130 and 140 are placed to measure a bioimpedance BIOZ of interest between nodes C and D of the human body or between electrodes 130 and 140. The generated voltage vin is applied to and evaluated by an evaluation circuit 300 which is illustrated in simplified form as a differential amplifier or instrumentation amplifier 310 in FIG. 1. The amplitude and phase of the resulting voltage signal vin depends on the bioimpedance BIOZ of interest, and the bioimpedance BIOZ is the quotient vin/iin.

Ideally, the voltage vin at the input of the evaluation circuit 300 is equal to the desired differential signal vdm which represents the voltage drop across the bioimpedance BIOZ. In reality, however, due to asymmetries in the current path a common mode signal is created across the body impedance BIOZ. Assuming that in some implementations, the common mode at nodes A and B is regulated to be some internal defined reference voltage vref, the common mode signal across nodes C and D is:

$$\frac{V C + V D}{2} = \frac{V A + VB}{2} + \frac{Z 4 - Z 3}{2} \cdot iin = vref + k \cdot iin$$

where VA is the voltage potential at node A, VB is the voltage potential at node B, VC is the voltage potential at node C, and VD is the voltage potential at node D.

As a result, a common mode signal vcm is generated across the bioimpedance BIOZ between nodes C and D, wherein the common mode signal vcm is proportional to the stimulus current:

$$vcm = \frac{Z 4 - Z 3}{2} \cdot iin$$

Due to asymmetries in the voltage path, the common mode signal vcm is translated to an unwanted extra differential signal vdmx. Those asymmetries arise because of mismatch between the skin/electrode contact impedances.

For example, with R110, R120 being the resistive portions of electrode contact impedances Z1 and Z2, respectively, cin1, cin2 being input capacitances at the inputs of amplifier 310 and s being the Laplace parameter, vdmx results to

$$vdmx = ( \frac{1}{1 - R 110 \cdot cin 1 \cdot s} - \frac{1}{1 + R 120 \cdot cin 2 \cdot s} ) \cdot vcm .$$

It can be seen that the unwanted extra differential signal vdmx is frequency dependent due to the Laplace parameter s.

Input capacitances cin1, cin2 include parasitic capacitances that may vary with the individual contact of the electrodes at the human body. The unwanted extra differential signal vdmx can is reduced if R110·cin1 and R120·cin2 are matching. Such matching can be accomplished by respective adjustment of the first and the second compensation impedance ZC1, ZC2, which contribute to the resulting impedances at the amplifier input.

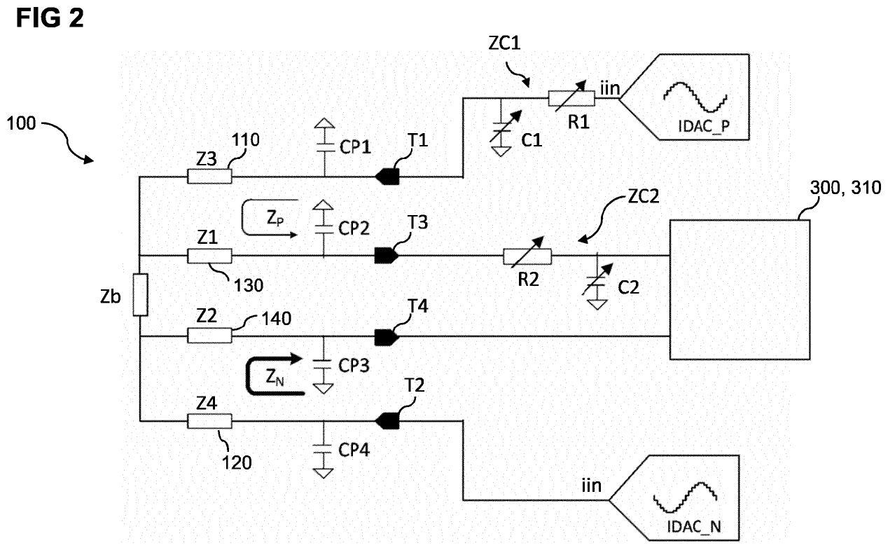

Referring now to FIG. 2, a further example implementation of a bioimpedance measurement circuit is shown, which is similar to the implementation of FIG. 1 and shows parasitic capacitances CP1, CP2, CP3, CP4 at the terminals of the bioimpedance measurement circuit. Hence, between the first terminal T1 and the third terminal T3 a first total impedance ZP results, corresponding to a first bodypart impedance. Similarly, a second total impedance ZN results between the second and the fourth terminal T2, T4 corresponding to a second bodypart impedance. If these impedances ZP, ZN do not match, the unwanted extra differential signal vdmx results. However, with the first and the second compensation impedance ZC1, ZC2 being present and adjusted accordingly, a mismatch of the resulting impedances at the input of the amplifier 310 can be reduced or even eliminated. For example, it is desired that a combination of the first total impedance ZP with the first and the second compensation impedance ZC1, ZC2 corresponds to the second total impedance ZN. Herein it is assumed that ZN is larger than ZP. If ZP was larger than ZN, the terminals could be exchanged, or one or more further compensation impedances could be provided at the second and fourth terminal T2, T4.

For example, the first compensation impedance ZC1 comprises a first adjustable compensation resistance R1 and a first adjustable compensation capacitance C1, and the second compensation impedance ZC2 comprises a second adjustable compensation resistance R2 and a second adjustable compensation capacitance C2. The compensation resistances R1, R2 are placed in the current path to and from the terminals.

The compensation capacitances C1, C2 connect the current paths to ground or any other reference potential of the amplifier 310. It should be noted that in various implementations one or more of the compensation resistances R1, R2 and the compensation capacitances C1, C2 could be left out, leaving at least one of them. For example, in a specific implementation, only compensation resistance R1 and compensation capacitance C2 are present.

The current sources for example are formed from current digital to analog converters, also known as IDACs.

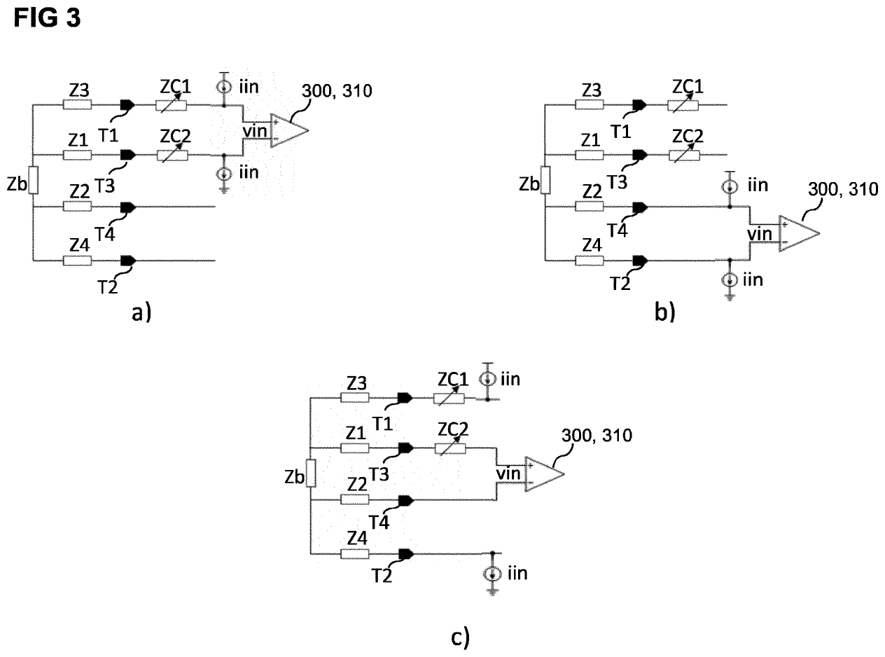

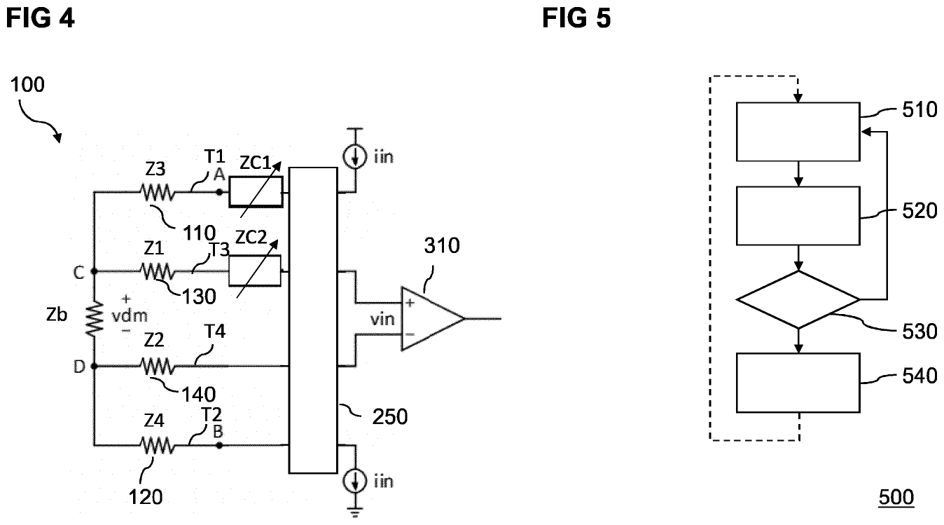

FIG. 3 shows various example measurement configurations a) and b) for a calibration mode of operation, and c) for a regular mode of operation of a bioimpedance measurement circuit. The respective configurations a) to c), for example can be achieved employing a multiplexer 250 as shown in FIG. 4. The multiplexer 250 is controlled by the control circuit 200, for example.

For example, in configuration a) for measuring a first bodypart impedance or wrist impedance, the stimulus current iin is applied through the first and the third terminal T1, T3 via the first and the second compensation impedance ZC1, ZC2, and the input voltage vin is measured between the first and the third terminal T1, T3. Similarly, in configuration b) for measuring a second bodypart impedance or finger impedance, the stimulus current iin is applied through the second and the fourth terminal T2, T4, and the input voltage vin is measured between the second and the fourth terminal T2, T4 or the corresponding electrodes, respectively. In configuration c) for measuring a body impedance, the stimulus current iin is applied through the first and the second terminal T1, T2, and the input voltage vin is measured between the third and the fourth terminal T3, T4 or the corresponding electrodes, respectively.

Hence with the measurements in configurations a) and b) it can be determined whether the resulting impedance ZP between terminals T1 and T3 matches the impedance ZN between terminals T4 and T2. Generally speaking, if such matching is achieved, an actual measurement of the bioimpedance Zb can be performed, inter alia involving the measurement in configuration c).

For example, the impedances ZP, ZN could be measured in configurations a) and b) while the compensation impedance is set to zero, and a desired value of the compensation impedance could be determined analytically from the measured impedances ZP, ZN.

However, the settings of the compensation impedances ZC1, ZC2 can be adjusted gradually by checking whether or how well a matching of the impedances between T1, T3 and T4, T2 is achieved in an iterative fashion. For example, FIG. 5 shows a method 500 for bioimpedance measurement that, e.g., is performed with the arrangement shown in FIG. 4. In a step 510, a first bodypart impedance between terminals T1 and T3 is determined, where the stimulus current iin is applied through the first and the third terminal T1, T3 and the compensation impedances ZC1, ZC2, and where the input voltage vin is measured between the first and the third terminal T1, T3. This corresponds to configuration a) of FIG. 3. For example, the first bodypart impedance corresponds to impedance ZP. In a step 520, a second bodypart impedance is determined between terminals T4 and T2, where the stimulus current iin is applied through the second and the fourth terminal T2, T4, and where the input voltage vin measured between the second and the fourth terminal T2, T4. For example, the second bodypart impedance corresponds to impedance ZN. It goes without saying that the order of steps 510 and 520 can be exchanged.

In step 530 a difference between the first and the second bodypart impedance is determined, e.g. by subtracting the bodypart impedances or by determining a ratio. The result can be a complex value. If a calibration criterion is not yet met, the compensation impedance ZC or ZC1, ZC2 is adjusted based on the difference and it is returned to step 510. For example, the calibration criterion can be a threshold, which the difference falls below. In addition or as an alternative, the calibration criterion can be a number of iterations performed, during which the bodypart impedances are determined and the calibration compensation impedance is adjusted. Steps 510, 520 and 530 are performed in a calibration mode of operation.

In step 540, which is performed in a regular mode of operation, the body impedance Zb is determined, where the stimulus current iin is applied through the first and the second terminal T1, T2, and where the input voltage vin is measured between the third and the fourth terminal T3, T4. This corresponds to configuration c) of FIG. 3. Further measurements can be performed in the regular mode of operation, if for example required for additional analytical compensation of remaining mismatch in the contact impedances. Furthermore, the body impedance Zb can be determined repetitively and/or consecutively in the regular mode of operation, once the compensation impedance ZC or ZC1, ZC2 has been finally adjusted in the calibration mode of operation.

If a new measurement cycle with a plurality of consecutive measurements in the regular mode of operation is to be started, where, for example, one or more of the electrodes 110, 120, 130, 140 have been moved, the whole process can be started over by returning to step 510, marked by the dashed arrow in FIG. 5, and performing a new calibration of the compensation impedance ZC or ZC1, ZC2.

The calibration in steps 510, 520 and 530 can be performed, for example, with one of a successive approximation approach and a binary search approach. In such approach the values of the adjustable portions of the compensation impedance ZC or ZC1, ZC2 are adjusted with the largest steps in the beginning and the smallest steps in the end, wherein with each adjustment step one “half” of the range corresponding to the current step is eliminated. For example, if the compensation impedance ZC is digitally controllable and the resulting values are adjusted with a digital control word, the calibration process starts with the most significant bit of the control word and proceeds towards the least significant bit.

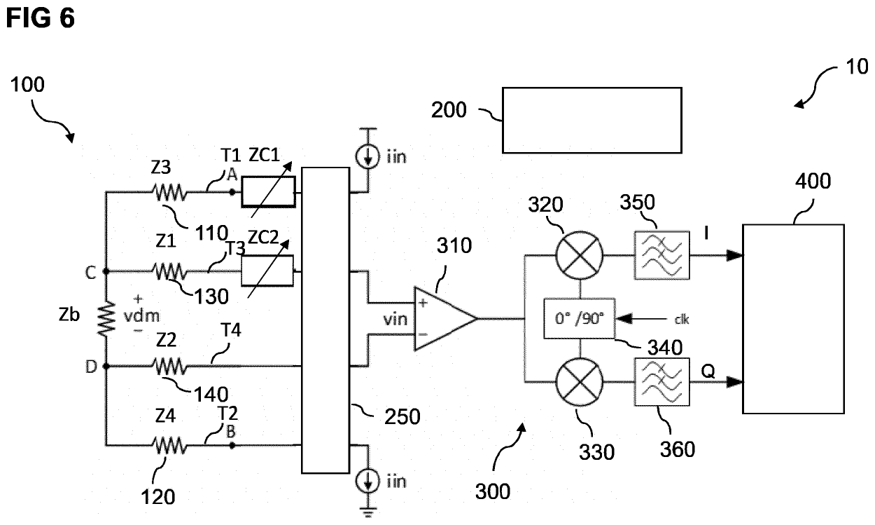

A bioimpedance measurement circuit 10 which allows to determine the real part I and the imaginary part Q of a measured bioimpedance is shown in FIG. 6, which is based on FIG. 4. The measurement circuit 10 comprises an electrode arrangement 100 for attaching to a body. The electrode arrangement 100 includes four electrodes 110, 120, 130, 140 to apply a stimulus current iin, and to measure an input voltage vin, depending on a configuration selected via multiplexer 250. The electrodes 110, 120 are attached to nodes A and B of a human body, wherein Z3 denotes an electrode contact impedance between the skin of the body and the electrode 110, and Z4 denotes an electrode contact impedance between the skin of the body and the electrode 120.

The bioimpedance measurement circuit 10 further comprises a control circuit 200 to control respective impedance measurements, and an evaluation circuit 300. The evaluation circuit 300 is configured for determining a real part I and an imaginary part Q of the measured impedance in response to the stimulus current iin and the measured input voltage vin.

The evaluation circuit 300 comprises a differential amplifier 310 to apply the measured input voltage vin. An output side of the differential amplifier 310 is coupled to a first path comprising a modulator 320 and a low pass filter 350, and a second path comprising a modulator 330 and a low pass filter 360. The modulators 320 and 330 are coupled to an oscillator 340. The arrangement of modulators 320, 330 coupled to oscillator 340 with low pass filters 350, 360 arranged behind the modulators 320, 330 is provided to implement a quadrature demodulation which allows to measure the real part I and the imaginary part Q of the measured impedance.

Referring to FIG. 6, the bioimpedance measurement circuit comprises a signal processing circuit 400, e.g. a processor. To this end the signal processing circuit 400 may comprise or be connected to a memory for storing values.

The control circuit 200 is configured to control the measurement of the impedances by applying the stimulus current iin with a measurement frequency F to the electrode arrangement 100, in particular by controlling the multiplexer 250 according to the desired impedance.

The control circuit 200 may also control the different operating modes, i.e. calibration operation and regular operation or measurement operation, respectively. The control circuit 200 itself may be controlled by the signal processing circuit 400.

The embodiments of the improved bioimpedance measurement concept disclosed herein have been discussed for the purpose of familiarizing the reader with novel aspects of the implementation of the improved bioimpedance measurement concept. Although various embodiments have been shown and described, many changes, modifications, equivalents and substitutions of the disclosed concepts may be made by one having skill in the art without departing from the scope of the claims.

In particular, the implementation of the improved bioimpedance measurement concept is not limited to the disclosed embodiments, and gives examples of many alternatives possible for the features included in the embodiments discussed. However, it is intended that any modifications, equivalents and substitutions of the disclosed concepts be included within the scope of the claims which are appended hereto.

Features recited in separate dependent claims may be advantageously combined. Moreover, reference signs used in the claims are not limited to be construed as limiting the scope of the claims.

Furthermore, as used herein, the term “comprising” does not exclude other elements. In addition, as used herein, the article “a” is intended to include one or more than one component or element, and is not limited to be construed as meaning only one.

1. A bioimpedance measurement circuit, comprising
a set of terminals comprising
a first terminal for connecting a first electrode to be attached to a body;
a second terminal for connecting a second electrode to be attached to the body;
a third terminal for connecting a third electrode to be attached to the body;
a fourth terminal for connecting a fourth electrode to be attached to the body;
a compensation impedance formed from at least one of a first compensation impedance connected to the first terminal and a second compensation impedance connected to the third terminal;
a control circuit to control application of a stimulus current with a measurement frequency through a first subset of two selected terminals of the set of terminals and measurement of an input voltage in response to the stimulus current at a second subset of two selected terminals of the set of terminals; and
an evaluation circuit for determining a measured impedance in response to the stimulus current and the measured input voltage;
wherein the control circuit is configured, in a calibration mode of operation,
to control measurement of a first bodypart impedance, where the stimulus current is applied through the first and the third terminal and the compensation impedance, and the input voltage is measured between the first and the third terminal;
to control measurement of a second bodypart impedance, where the stimulus current is applied through the second and the fourth terminal, and the input voltage is measured between the second and the fourth terminal; and
to adjust the compensation impedance based on a difference between the first bodypart impedance and the second bodypart impedance until a calibration criterion is met;
wherein the control circuit is further configured, in a regular mode of operation, to control measurement of a body impedance, where the stimulus current is applied through the first and the second terminal, and the input voltage is measured between the third and the fourth terminal.

2. The bioimpedance measurement circuit according to claim 1, wherein the compensation impedance comprises at least one of a resistive portion and a capacitive portion.

3. The bioimpedance measurement circuit according to claim 1, wherein the compensation impedance is digitally controllable, and wherein the control circuit is configured to adjust the compensation impedance with a digital control word.

4. The bioimpedance measurement circuit according to claim 1, wherein the calibration criterion is at least one of a number of iterations and a difference threshold for the difference between the first bodypart impedance and the second bodypart impedance.

5. The bioimpedance measurement circuit according to claim 1, wherein the control circuit is configured, in the calibration mode of operation, to perform one of a successive approximation approach and a binary search approach.

6. The bioimpedance measurement circuit according to claim 1, which is configured to perform a measurement cycle with a plurality of consecutive measurements in the regular mode of operation, wherein each measurement cycle is preceded by a calibration in the calibration mode of operation.

7. The bioimpedance measurement circuit according to claim 1, further comprising a signal processing circuit for determining, from the corresponding measurements, the body impedance, the first bodypart impedance and the second bodypart impedance.

8. The bioimpedance measurement circuit according to claim 1, further comprising an electrode arrangement for attaching to a body, the electrode arrangement including the first electrode, the second electrode, the third electrode and the fourth electrode respectively connected to the corresponding terminal of the set of terminals.

9. An electronic device comprising a bioimpedance measurement circuit according to claim 1.

10. A bioimpedance measurement method being performed with a set of terminals comprising
a first terminal connected to a first electrode being attached to a body;
a second terminal connected to a second electrode being attached to the body;
a third terminal connected to a third electrode being attached to the body; and
a fourth terminal connected to a fourth electrode being attached to the body; and
with a compensation impedance formed from at least one of a first compensation impedance connected to the first terminal and a second compensation impedance connected to the third terminal;
the method comprising:
generating a stimulus current with a measurement frequency;
determining, in a calibration mode of operation, a first bodypart impedance in response to the stimulus current and the measured input voltage, where the stimulus current is applied through the first and the third terminal and the compensation impedance, and the input voltage is measured between the first and the third terminal;
determining, in the calibration mode of operation, a second bodypart impedance in response to the stimulus current and the measured input voltage, where the stimulus current is applied through the second and the fourth terminal, and the input voltage is measured between the second and the fourth terminal;
adjusting, in the calibration mode of operation, the compensation impedance based on a difference between the first bodypart impedance and the second bodypart impedance until a calibration criterion is met; and
determining, in a regular mode of operation, a body impedance in response to the stimulus current and a measured input voltage in response to the stimulus current, where the stimulus current is applied through the first and the second terminal, and the input voltage is measured between the third and the fourth terminal.

11. The method according to claim 10, wherein the compensation impedance comprises at least one of a resistive portion and a capacitive portion.

12. The method according to claim 10, wherein the compensation impedance is digitally controllable, and wherein adjusting the compensation impedance is performed with a digital control word.

13. The method according to claim 10, wherein the calibration criterion is at least one of a number of iterations and a difference threshold for the difference between the first bodypart impedance and the second bodypart impedance.

14. The method according to claim 10, wherein, in the calibration mode of operation, one of a successive approximation approach and a binary search approach is performed.

15. The method according to claim 10, further comprising performing a measurement cycle with a plurality of consecutive measurements in the regular mode of operation, wherein each measurement cycle is preceded by a calibration in the calibration mode of operation.

16. A computer program product comprising instructions which, when executed on one or more processors in connection with a first, a second, a third, a fourth terminal and a compensation impedance, cause the one or more processors to perform the bioimpedance measurement method according to claim 10.
